(19) United States
(12) Patent Application Publication
(10) Pub. No.: US 2023/0048392 A1
(43) Pub. Date: Feb. 16, 2023

(54) MICROPIGMENTATION DEVICE AND METHOD OF USE

(71) Applicant: Micropigmentation Academy LLC, Wethersfield, CT (US)

(72) Inventor: Anthony E. Angelillo, New Britain, CT (US)

(21) Appl. No.: 17/886,454

(22) Filed: Aug. 11, 2022

Related U.S. Application Data
(60) Provisional application No. 63/232,077, filed on Aug. 11, 2021.

(51) Int. Cl. A61M 37/00; A61B 17/34

(52) U.S. Cl. CPC A61M 37/0084; A61B 17/34; A61B 2017/00747

(57) ABSTRACT

A micropigmentation device and related method of use may be used for micropigmentation and other similar procedures. The micropigmentation device may include two needle tips, which may optionally each be part of a separate needle or may be tips on a single component, such as a single needle. In other embodiments, the device may include pairings of needle tips, such as two components with each having a plurality of needle tips or a plurality of needles grouped into two groupings of needle tips. The device may be used to simulate natural human hair follicles as they grow in pairs.

### BACKGROUND

This application claims priority to U.S. Provisional Pat. Application No. 63/232,077 filed on Aug. 11, 2021, the disclosure of which is incorporated herein in its entirety.

The present disclosure relates generally to a piercing device and method of use. More particularly, the present disclosure relates to a device for piercing, such as for dermal piercing or tattooing, which may be used as part of a system with micropigmentation equipment or may itself be a feature of such equipment, and associated methods of use.

Micropigmentation is a form of what is commonly referred to as “permanent makeup”. It is also commonly referred to as dermapigmentation or cosmetic tattooing. Micropigmentation uses permanent or semi-permanent pigmentation of the dermis, also known as “tattooing”, to leave desired marks and colors on the skin. Micropigmentation may be used to disguise what an individual may consider to be imperfections in their appearance, or it may be used to enhance their appearance to desired levels. In one application of micropigmentation, an individual may desire to have the appearance of additional hair follicles, or a different pattern, density, or coloring of hair follicles. Common applications of micropigmentation to simulate hair follicles include eyebrows, such as for those who have lost their eyebrows due to medical reasons or simply desire fuller or differently shaped eyebrows, as well as hairlines, hair density, and undesirably bald areas. One who applies micropigmentation, such as a tattoo artist or a micropigmentation technician or specialist, may create the appearance of hair follicles on an individual through the use of micropigmentation, applying substances such as tattoo ink to the dermis or below the dermis at desired depths, desired angles, in desired patterns, and in desired colors.

Various devices and methods related to micropigmentation are known in the art. Most means of applying micropigmentation involve the use of one of a number of types of tools, such as tattoo guns, which use a needle of varying gauges to apply the pigmentation. Needles, as well as the associated cartridges and also ink/pigmentation reservoirs, are typically disposable, with a new one being used for each customer, thus reducing the risk of cross-contamination between customers, or whenever previous components otherwise require replacement. The applicator is commonly handheld, requiring the micropigmentation specialist to maintain position, angle, and depth of needle penetration completely by hand and by eye.

A common problem with micropigmentation is when a user is attempting to realistically simulate hair follicles, particularly in creating groupings which are even, consistent, and resemble actual hair follicles upon close inspection or otherwise at a close distance to the skin. Users are currently restricted to needle cartridges which often include five or seven needles, thus creating a larger pigmented area which only appears as hair from a substantial distance from the skin. In some cases, users may use only a single needle, thus having to attempt to show follicles at realistic distances from other follicles and, when dealing with pairings of follicles, trying to group points of pigmentation extremely closely while also appearing as distinctive pairings of follicles. This is a time-consuming and, most commonly, very inconsistent process that leads to unrealistic results and difficult micropigmentation procedures.

Current micropigmentation devices and methods of use continue to lead to imperfect results even in the hands of skilled micropigmentation specialists. Current micropigmentation devices do not adequately enable even, consistent, and realistic application of micropigmentation at controlled and repeatable patterns.

### SUMMARY

The present disclosure relates generally to a piercing device and method of use. More particularly, the present disclosure relates to a device for piercing, such as for dermal piercing or tattooing, which may be used as part of a system with micropigmentation equipment or may itself be a feature of such equipment, and associated methods of use.

The micropigmentation device may include a first needle tip configured to apply a liquid through a hole in a material and defining a first axis and a second needle tip configured to apply a liquid through a hole in a material and defining a second axis. The first first axis and the second axis may optionally be substantially parallel, though it should be understood that they may be angled relative to each other, such as to create improved visibility or ink application while maintaining piercing points in the material that are proximate to each other. The material may be a dermis, such as human skin.

The liquid that is applied may be ink, such as used for micropigmentation, or may be other liquids, such as medication or a carrier for medication, or carriers for antivirals or other medically applied liquids.

The micropigmentation device may be part of a tattoo gun or other micropigmentation device, or may be removably connected to such a device.

The first needle tip and the second needle tip may be connected. One or both of the needle tips may be a feature of a needle. The two needles may be unitary, such as where they have been formed by extrusion or molding or other means known in the art, or may be connected by means of welding, adhesives, or other means known in the art.

The first needle may be connected to the second needle by means of a cartridge, the tattooing device, a bar or armature, or other features or components. The two needles may be connected by means of a sleeve or a secondary element, such as a larger needle or cylindrical shape, though other shapes may also be used without deviating from the scope or intent of this disclosure.

In one embodiment, the micropigmentation device comprises exactly two needles, though it should be understood that other embodiments may include pairings of other quantities of needles, such as a pair of needles which each include a plurality of smaller needle tips, or groupings of needles into two groupings, so as to create substantially two entry points into the dermis or to leave ink or other markings to simulate two marks or dots.

A method of applying pigmentation below a dermis may include using a micropigmentation device that further comprises a first needle tip and a second needle tip. The device may comprise exactly two needle tips, or may include pairings of other quantities of needles, such as a pair of needles which each include a plurality of smaller needle tips, or groupings of needles into two groupings, so as to create substantially two entry points into the dermis or to leave ink or other markings to simulate two marks or dots. The method comprises the creating of two marked points or areas proximate to each other, thus creating the appearance of pairings of hair follicles.

In one embodiment, the axis defined by the first needle of the device is spaced relative to the axis defined by the second needle of the device at such as distance as to approximate or simulate the distance between human hair follicles. When the two needles move in unison, relative to a cowling of the device, they may apply pigmentation to or below the dermis of a patient so as to simulate the spacing between pairs of hair follicles.

In one method of use, the needle cartridge may be circular in cross-section or otherwise make it difficult for a user to locate the plane of the two needles. In such an embodiment, a user may extend the needles from the needle cartridge and then mark a location on the needle cartridge or on a component of the device so as to enable location of the two needle tips during use more easily. The needles may then be retracted in preparation for use. In another embodiment, the device or the cartridge may include a feature or marking to show the location or plane of the needles. In other embodiments, the needle cartridge may only connect to the micropigmentation device in an orientation to enable a user to more readily determine the orientation of the needles, such as where the device may have markings or features for orientation.

In some embodiments, the needle cartridge may include one or more features to aid in orientation of the needles or to aid a user in visualizing the orientation of the needles or a plane extending between the needles. In some embodiments, the needle cartridge may be shaped to enable this, such as where the needle cartridge may be rectangular or have a feature for orientation or visualization. In other embodiments, the needle pair may extend from a slot or the components of the micropigmentation device may include a slot or other orientation feature or opening.

The disclosure relates to a micropigmentation device comprising two needle tips and method of use, or of two groupings of needle tips and method of use, such as through the use of the described invention. The method may include the tattooing or otherwise micropigmenting in pairings of needle locations so as to simulate natural human hair follicular growth. Any number of components or features may be used in conjunction with and incorporated into the micropigmentation device described. Various materials, including metals, plastics, and composites, may be used for the described components and features.

It is to be understood that the above mentioned features and the features yet to be explained hereinafter can be used not only in the respectively mentioned combinations but also in other combinations or alone without departing from the context of the present invention.

### BRIEF DESCRIPTION OF THE DRAWINGS

The invention is now disclosed in detail with reference to exemplary embodiments shown in the accompanying drawings, where:

FIG. 1 shows a front top right isometric view of an embodiment of a micropigmentation device of the present disclosure;

FIG. 2 shows a detail view of the embodiment of the micropigmentation device of FIG. 1;

FIG. 3 shows a front view of an embodiment of the micropigmentation device of the present disclosure;

FIG. 4 shows a detail view of the embodiment of the micropigmentation device of FIG. 3;

FIG. 5 shows a right side view of an embodiment of the micropigmentation device of the present disclosure;

FIG. 6 shows a detail view of the embodiment of the micropigmentation device of FIG. 5;

FIG. 7 shows a front right top isometric view of an embodiment of the micropigmentation device of the present disclosure;

FIG. 8 shows a bottom view of an embodiment of the micropigmentation device of the present disclosure; and

FIG. 9 shows a photograph of a front view of an embodiment of the micropigmentation device of the present disclosure.

### DETAILED DESCRIPTION

Exemplary embodiments of the disclosure are illustrated in the Figures and are explained in the following description in more detail, wherein identical reference numbers refer to identical, or similar, or functionally identical or similar components.

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features or those previously described are omitted or simplified in order not to obscure the illustrative embodiments.

Various operations may be described as multiple discrete operations, in turn, in a manner that is most helpful in understanding the present invention, however the order of description should not be construed as to imply that these operations are necessarily order dependent. In particular, these operations need not be performed in the order of presentation. In some instances, structures and devices may be shown in block diagram or flow chart form in order to facilitate describing the disclosed subject matter.

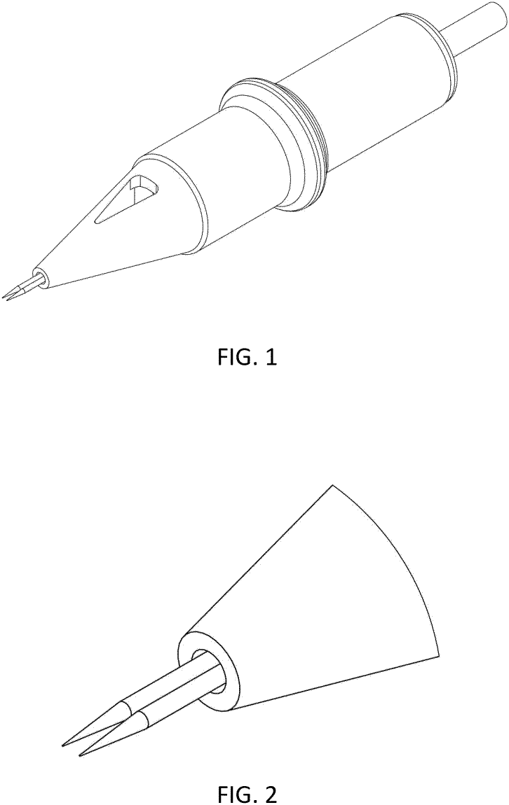

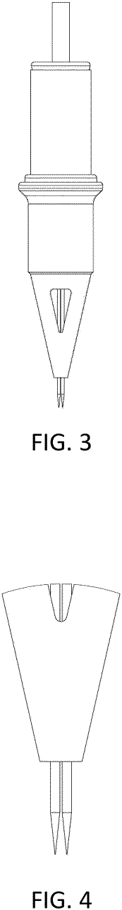

An embodiment of the micropigmentation device of the present disclosure is shown in FIGS. 1-2. As shown, the device includes a cartridge for tattooing or micropigmenting, which may optionally include ink. The cartridge may optionally be attachable, such as in a removable fashion, to a tattoo gun or other micropigmentation tool. The micropigmentation device includes two needs which can be seen extending from a bottom end of the cartridge, through an opening, though it should be understood that the needles may be substantially exposed, substantially enclosed, or may be completely exposed without deviating from the scope or intent of this disclosure.

The two needles may be grouped and located proximal to each other, so as to simulation a pair of hair follicles. One or both of the needles may comprise tips which may be substantially flat, substantially angled, conical or pointed, or other shapes known in the art. The needles are shown substantially circular in cross-section, though it should be understood that other shapes of needles may also be used without deviating from the scope or intent of this disclosure. The needles may also be hollow, solid, or have partial cutaways, grooves, notches, slots, or may include other features or irregularities. One or both needles may pierce a material, such as a dermis, and deliver ink, such as by pushing ink down into an opening with the one or both needles, or may contain an opening, such as a slot passing at least partially through the needles, through which ink may pass. It should be understood that other fluids may also be used, including ink, water, saline, dyes, medications, and other things known in the art.

Though not shown, the needles may be completely separate, connected by means of the cartridge or by other components or features of a tattoo gun or other pigmentation device. The needles may also be welded to each other or adhered through sintering or other means known in the art. The needles may also be connected by means or an armature or bar. The needles may also be connected by means of a sleeve. The needles may also be formed unitary, such as being extruded together, forged, or created through other means known in the art. The needles may also be a substantially unitary component where two ends are formed, such as by polishing or machining, creating two needle tips while being a substantially singular component.

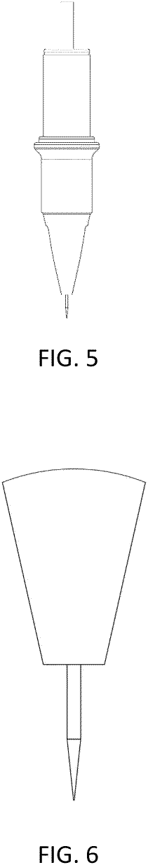

FIGS. 3-4 show a front view of the micropigmentation device of the present disclosure. The needles can be seen side-by-side, substantially within a single plane. As can be seen in the embodiment shown in FIGS. 5-6, a side view with both needles substantially within a single plane shows the needles to be aligned.

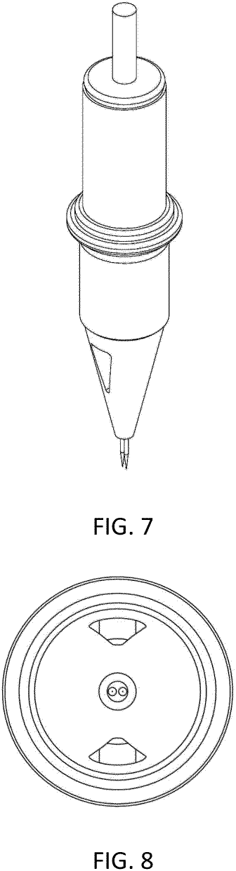

An isometric view of the micropigmentation device of the present disclosure is shown in FIG. 7, with a bottom view shown in FIG. 8.

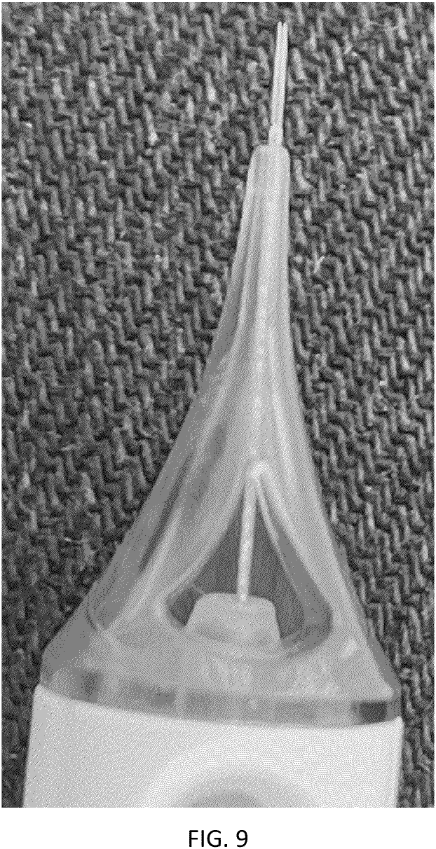

As can be seen in the photograph in FIG. 9, the end of the micropigmentation device may show two needle tips, while the bottom of a needle assembly extending into a cartridge or other micropigmentation device may show a welded or otherwise substantially unitary construct which terminates in two needle tips.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention. Accordingly, various modifications, adaptations, combinations, and alternatives may occur to one skilled in the art without departing from the spirit of the invention and scope of the claimed coverage.

What is claimed is:

1. A micropigmentation device, comprising:
a first needle tip configured to apply a liquid through a hole in a material and defining a first axis; and
a second needle tip configured to apply a liquid through a hole in a material and defining a second axis;
wherein the first axis and the second axis are substantially parallel, and further wherein the first needle tip is configured to translate relative to a cowling of the micropigmentation device along the first axis, the second needle tip is configured to translate relative the cowling of the micropigmentation device along the second axis, and the first needle tip and the second needle tip are configured to translate in unison.

2. The micropigmentation device according to claim 1, further wherein the material is a dermis.

3. The micropigmentation device according to claim 1, further wherein the liquid is ink.

4. The micropigmentation device according to claim 1, further wherein the first axis and the second axis define a spacing distance between the first axis and the second axis, and further wherein the spacing distance approximates the distance between human hair follicle pairs.

5. The micropigmentation device according to claim 1, further wherein the micropigmentation device is removably connectable to a tattoo gun.

6. The micropigmentation device according to claim 1, further wherein the first needle tip and the second needle tip are connected.

7. The micropigmentation device according to claim 1, further wherein the first needle tip is a feature of a first needle and the second needle tip is a feature of a second needle.

8. The micropigmentation device according to claim 7, further wherein the first needle is unitary with the second needle.

9. The micropigmentation device according to claim 7, further wherein the first needle is connected to the second needle.

10. The micropigmentation device according to claim 9, further wherein the first needle is connected to the second needle by means of a cartridge.

11. The micropigmentation device according to claim 9, further wherein the first needle is connected to the second needle by means of a bar.

12. The micropigmentation device according to claim 9, further wherein the first needle is welded to the second needle.

13. A method of applying pigmentation below a dermis, comprising:
using a micropigmentation device that further comprises a first needle tip and a second needle tip;
wherein the pairs of pigmentation applications are applied below the dermis.

14. The micropigmentation device according to claim 1, further wherein the micropigmentation device comprises exactly two needle tips.

15. The micropigmentation device according to claim 1, further wherein the micropigmentation device comprises exactly two needles.

16. A method of applying pigmentation at or below a dermis, comprising:
connecting an ink cartridge to a micropigmentation device;
connecting a needle cartridge to the micropigmentation device; and
using the micropigmentation device to simulate hair follicle pairs on or below a patient’s dermis;
wherein the micropigmentation device further comprises two needles which are configured to at least partially extend from a housing during use of the micropigmentation device, and further wherein the two needles translate along a first axis defined by a first needle and a second axis defined by a second needle in unison.

17. The method of method of applying pigmentation at or below a dermis according to claim 16, further wherein the micropigmentation device comprises exactly two needle tips.

18. The method of method of applying pigmentation at or below a dermis according to claim 16, further wherein the needle cartridge and the ink cartridge or unitary.

19. The method of method of applying pigmentation at or below a dermis according to claim 16, further comprising extending the two needles from a housing of the needle cartridge, at least partially retracting the two needles at least partially within the housing, and marking the location of a plane extending between a first axis of a first needle and a second axis of a second needle on a component of the micropigmentation device.

20. The method of method of applying pigmentation at or below a dermis according to claim 16, further wherein the micropigmentation devices comprises a mark or feature configured to denote a plane extending between a first axis of a first needle and a second axis of a second needle.
